(19) United States
(12) Patent Application Publication
(10) Pub. No.: US 2025/0384551 A1
(43) Pub. Date: Dec. 18, 2025

(54) METHOD AND SYSTEM FOR GENERATING CARDIAC MECHANICAL SIGNALS

(71) Applicant: St. Jude Medical, Cardiology Division, Inc., St. Paul, MN (US)

(72) Inventors: Lorina Dascal, Haifa (IL); Avi Motova, Haifa (IL); Jacob Segev, Haifa (IL)

(21) Appl. No.: 19/229,587

(22) Filed: Jun. 5, 2025

Related U.S. Application Data
(60) Provisional application No. 63/661,316, filed on Jun. 18, 2024.

(51) Int. Cl. G06T 7/00; A61B 8/00; A61B 8/04; A61B 8/08; G06T 7/11; G06T 7/62

(52) U.S. Cl. CPC G06T 7/0012; A61B 8/04; A61B 8/0883; A61B 8/5207; G06T 7/11; G06T 7/62; G06T 7/97; G06T 2200/04; G06T 2207/10136; G06T 2207/30048

(57) ABSTRACT

An electroanatomical mapping system generates a cardiac mechanical signal from a plurality of cardiac images and, more particularly, a plurality of ultrasound images. For each image, the system segments a blood pool/tissue border to detect a blob and defines a size metric, such as volume, area, maximum axial dimension, Zernike Moment, or the like for the detected blob. The plurality of size metrics thus generated can be output as a function of time to generate the cardiac mechanical signal. Local minima in the signal correspond to systole, while local maxima in the signal correspond to diastole.

### CROSS-REFERENCE TO RELATED APPLICATIONS

This application claims the benefit of U.S. provisional application No. 63/661,316, filed 18 Jun. 2024, which is hereby incorporated by reference as though fully set forth herein.

### FIELD

The present disclosure relates generally to medical procedures, such as cardiac diagnostic and therapeutic procedures, including electrophysiological mapping and cardiac ablation. In particular, the present disclosure relates to generating cardiac mechanical signals such as may be used as gating signals in such procedures.

### BACKGROUND

In connection with various cardiac diagnostic and therapeutic procedures, it is known to create a three-dimensional anatomical model of the heart chamber(s) being studied and/or a map of the electrical activity thereof. Those of ordinary skill in the art will recognize that such models and maps are often created by collecting numerous data points from the chamber(s) of interest.

It is desirable to synchronize data point collection with heart motion. Similar synchronization is desirable when collecting cardiac imagery (e.g., intracardiac echocardiographic (ICE) imagery). Typically, synchronization relies on the heart's electrical polarization and depolarization cycle, as measured by an electrocardiogram (ECG or EKG), as a gating signal for data acquisition (whether those data are geometry points for creation of a three-dimensional anatomical model, electrophysiology data points for creation of an electrophysiology map, and/or two- or three-dimensional ultrasound images).

Yet, an ECG signal is merely a proxy for, and may not be perfectly synchronized to, the heart's actual movement. Indeed, in a patient experiencing an arrythmia, the ECG signal may vary significantly from the heart's actual movement.

### BRIEF SUMMARY

The instant disclosure provides a method of generating a cardiac mechanical signal, including the steps of: receiving a plurality of cardiac images in an electroanatomical mapping system; for each cardiac image of the plurality of cardiac images, the electroanatomical mapping system identifying a cardiac area metric, thereby identifying a plurality of cardiac arca metrics; and outputting, via the electroanatomical mapping system, a trace of the plurality of cardiac area metrics as a function of time, thereby generating the cardiac mechanical signal.

The step of identifying a cardiac area metric for each cardiac image of the plurality of cardiac images can include the electroanatomical mapping system: segmenting a blood pool/tissue border in the respective cardiac image; detecting a blob in the segmented respective cardiac image; and defining the cardiac area metric for the respective cardiac image as a function of a characteristic of the detected blob. The characteristic of the detected blob can include an area of the detected blob, a volume of the detected blob, a maximum axial dimension of the detected blob, a Zernike Moment of the blob, or another characterization/quantification of the shape of an object.

The plurality of cardiac images can include a plurality of ultrasound images, such as a plurality of intracardiac echocardiography (ICE) images. It is contemplated that the plurality of ultrasound images may be two- and/or three-dimensional. They may be of a single heart chamber or of another anatomical region.

In additional aspects, the method further includes: the electroanatomical mapping system identifying a local minimum in the cardiac mechanical signal as a systole; and the electroanatomical mapping system identifying a local maximum in the cardiac mechanical signal as a diastole.

Also disclosed herein is an electroanatomical mapping system including a mechanical signal generation module configured to: receive as input a plurality of cardiac images; for each cardiac image of the plurality of cardiac images, identify a cardiac area metric, thereby identifying a plurality of cardiac area metrics; and output a trace of the plurality of cardiac area metrics as a function of time, thereby generating a cardiac mechanical signal.

The mechanical signal generation module can be configured to identify the cardiac arca metric for each image of the plurality of cardiac images by: segmenting a blood pool/tissue border in the respective cardiac image; detecting a blob in the segmented respective cardiac image; and defining the cardiac area metric for the respective cardiac image as a function of a characteristic of the detected blob. The characteristic of the detected blob can be an area of the detected blob, a volume of the detected blob, a maximum axial dimension of the detected blob, a Zernike Moment of the detected blob, or another characterization/quantification of the shape of the blob.

In another aspect of the disclosure, the mechanical signal generation module is further configured to: identify a local minimum in the cardiac mechanical signal as a systole; and identify a local maximum in the cardiac mechanical signal as a diastole.

The present disclosure also provides a method of generating a signal representative of variations in a size of an anatomical feature from a plurality of images of the anatomical feature. The method includes the steps of receiving, in a mechanical signal generation module, the plurality of images of the anatomical feature; and for each image of the plurality of images, the mechanical signal generation module: segmenting a boundary of the anatomical feature in the respective image; detecting a blob in the segmented respective image; and defining a size metric for the respective image as a function of a characteristic of the detected blob, thereby defining a plurality of size metrics; and outputting, via the mechanical signal generation module, a trace of the plurality of size metrics as a function of time, thereby generating the signal representative of variations in the size of the anatomical feature.

The size metric can be selected from the group consisting of an area of the detected blob, a volume of the detected blob, and a maximum axial dimension of the detected blob. Other size metrics, such as Zernike Moments, are also contemplated.

The method can further include at least one of: identifying a local minimum in the signal as a minimum size of the anatomical feature; and identifying a local maximum in the signal as a maximum size of the anatomical feature.

There is also provided a computer readable medium, a record carrier or a computer program product comprising instructions that, when executed, cause a computer or processor to perform any of the methods set forth herein.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 is a flowchart of representative steps that can be carried out according to aspects of the instant disclosure.

FIG. 3 depicts representative two-dimensional image slices of a cardiac chamber.

FIG. 4 depicts a representative blob, such as may be derived by segmentation of a two-dimensional image slice as shown in FIG. 3.

FIG. 5 is an illustrative cardiac mechanical signal according to the instant teachings.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for generating anatomical mechanical signals, such as cardiac mechanical signals, using time-sequenced medical images, such as ultrasound images of a subject's heart. Cardiac mechanical signals may be desirable, for example, as gating signals during an electrophysiology study. In this regard, cardiac mechanical signals may offer various advantages over cardiac electrical signals, such as ECG signals, which may be more difficult to correlate to cardiac mechanical function, particularly in subjects experiencing arrythmias.

For purposes of illustration, aspects of the disclosure will be described with reference to generating cardiac mechanical signals from a time series of two-dimensional ICE image slices. Two-dimensional ICE images may be collected using an ICE catheter, such as Abbott Laboratories' ViewFlex™ Xtra ICE catheter (Abbott Park, Illinois). Exemplary embodiments will further be described in the context of a procedure carried out using an electroanatomical mapping system, such as the EnSite Precision™ cardiac mapping system or the Ensite™ X EP System, both also from Abbott Laboratories.

Those of ordinary skill in the art will understand, however, how to apply the teachings herein to good advantage in other contexts and/or with respect to other devices. For instance, the ordinarily-skilled artisan will appreciate how to extend the teachings herein to three-dimensional (volumetric) images and/or to images of anatomical regions other than the heart. Likewise, the ordinarily-skilled artisan will appreciate that the teachings herein can be applied to other types of images, including, without limitation, transesophageal echocardiographic (TEE) images, transthoracic echocardiographic (TTE) images, and other acoustic images.

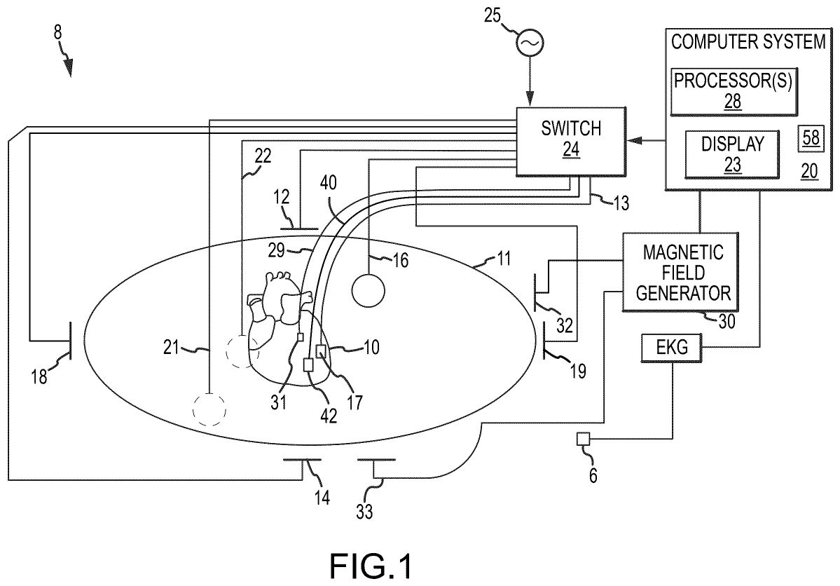

FIG. 1 shows a schematic diagram of an exemplary electroanatomical mapping system 8 for conducting cardiac electrophysiology procedures, such as electrophysiological mapping and ablation. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

As depicted in FIG. 1 and described herein, system 8 can be a hybrid system that incorporates both impedance-based and magnetic field-based localization capabilities. In some embodiments, system 8 is the EnSite™ Velocity™ or EnSite Precision™ cardiac mapping system or the Ensite™ X EP System, all from Abbott Laboratories. Other electroanatomical mapping systems, however, may be used in connection with the present teachings, including, for example, the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario, Canada), and Stereotaxis, Inc.'s (St. Louis, Missouri) NIOBE® Magnetic Navigation System.

The localization and mapping systems described in the following patents (all of which are hereby incorporated by reference in their entireties) can also be used with the instant teachings: U.S. Pat. Nos. 6,990,370; 6,978,168; 6,947,785; 6,939,309; 6,728,562; 6,640,119; 5,983,126; and 5,697,377.

The foregoing systems, and the modalities they employ to localize a medical device, will be familiar to those of ordinary skill in the art. Insofar as the ordinarily-skilled artisan will appreciate the basic operation of such systems, therefore, they are only described herein to the extent necessary to understand the instant disclosure.

For simplicity of illustration, the patient 11 is depicted schematically as an oval. In the embodiment shown in FIG. 1, three sets of surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments the electrodes could be positioned in other arrangements, for example multiple electrodes on a particular body surface. As a further alternative, the electrodes do not need to be on the body surface, but could be positioned internally to the body. Regardless of configuration, the patient's heart 10 lies within the electric field generated by patch electrodes 12, 14, 16, 18, 19, and 22.

FIG. 1 also depicts a magnetic source 30, which is coupled to magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrodes 12, 14, 16, 18, 19, and 22) can be used without departing from the scope of the present teachings.

An additional surface reference electrode (e.g., a “belly patch”) 21 provides a reference and/or ground electrode for the system 8. The belly patch electrode 21 may be an alternative to a fixed intra-cardiac electrode 31, described in further detail below. A magnetic patient reference sensor-anterior (“PRS-A”) can also be positioned on the patient's chest to serve as a reference, analogous to surface reference electrode 21 and/or intracardiac reference electrode 31, for magnetic field-based localization modalities.

It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

Representative catheters 13, 40 are also shown schematically in FIG. 1. In aspects of the disclosure, catheter 13 can be an ablation catheter, such as the Abbott Laboratories FlexAbility™ Ablation Catheter, Sensor Enabled™, and catheter 40 can be an intracardiac echocardiography (ICE) catheter, such as the Abbott Laboratories ViewFlex™ Xtra ICE catheter. Catheters 13, 40 each respectively include one or more sensors 17, 42 for sensing the electric fields generated by patch electrodes 12, 14, 16, 18, 19, and 22 and/or the magnetic fields generated by magnetic field generators 32, 33.

In some embodiments, an optional fixed reference electrode 31 (e.g., attached to a wall of the heart 10) is shown on yet another catheter 29. Often, reference electrode 31 is placed in the coronary sinus and defines the origin of a coordinate system with reference to which catheters 13, 40 can be localized by system 8.

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Amongst other things, computer system 8 can interpret measurements by sensors 17, 42 of the magnetic and/or electrical fields generated by magnetic field generators 32, 33 and patch electrodes 12, 14, 16, 18, 19, and 22, respectively, to determine the position and orientation of catheters 13, 40 within heart 10. The term “localization” is used herein to describe the determination of the position and orientation of an object, such as catheter 13, within such fields.

Ultrasound imaging catheter 40 can be used to collect a plurality of two-dimensional images of heart 11 using any of several echographic imaging modalities, such as B-mode ultrasound and color Doppler echocardiography. These two-dimensional images can, in some embodiments of the disclosure, be assembled into a three-dimensional volumetric image of heart 11 (or other anatomic structure) using various techniques, including those disclosed in United States patent application publication no. 2006/0241445 (which is hereby incorporated by reference as though fully set forth herein). It is contemplated that ultrasound imaging catheter 40 may be coupled to an ultrasound console, such as Abbott Laboratories' ViewMate™ Ultrasound Console, which may in turn be coupled to system 8. Alternatively, and for purposes of the disclosure herein, ultrasound imaging catheter 40 will be described as coupled directly to system 8, such that aspects of the disclosure can be carried out on processor(s) 28 of computer 20.

The foregoing discussion of ICE imaging is general, insofar as numerous aspects of ICE imaging, including the use of ICE imaging in connection with electrophysiology procedures, are well-understood by those of ordinary skill in the art and need not be described in detail herein. See, e.g., Enriquez et al., “Use of Intracardiac Echocardiography in Interventional Cardiology,” Circulation, Vol. 137, Issue 21, pp.2278-2294 (May 22, 2018). Thus, ICE imaging will only be described herein to the extent necessary to understand the instant disclosure.

As mentioned above, aspects of the disclosure relate to generating anatomical mechanical signals from medical images. System 8 can therefore include a mechanical signal generation module 58, which may be software based (e.g., a series of programming instructions executed on processor(s) 28 of computer 20), hardware-based (e.g., an application specific integrated circuit (ASIC)), or a combination thereof.

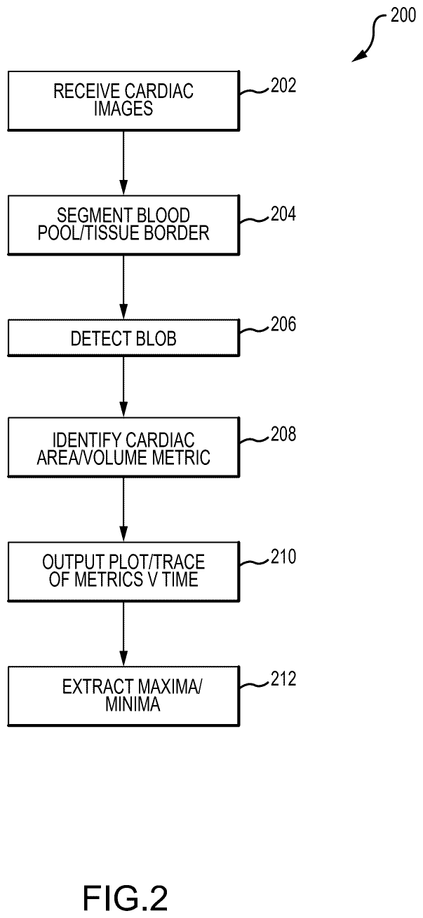

One exemplary method according to aspects of the instant disclosure will be explained with reference to the flowchart 200 of representative steps presented as FIG. 2. In some embodiments, for example, flowchart 200 may represent several exemplary steps that can be carried out by electroanatomical mapping system 8 of FIG. 1 (e.g., by processor(s) 28 and/or mechanical signal generation module 58). It should be understood that the representative steps described below can be either hardware- or software-implemented. For the sake of explanation, the term “signal processor” is used herein to describe both hardware- and software-based implementations of the teachings herein.

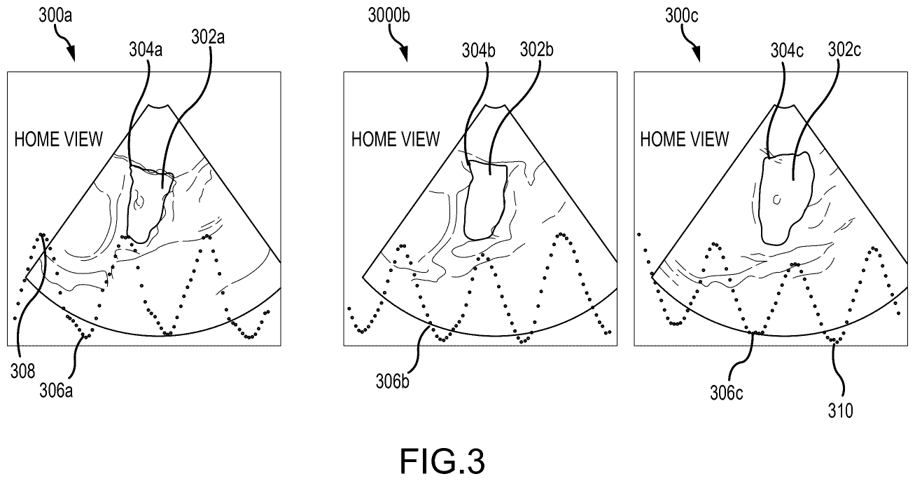

In block 202, system 8 receives a plurality of timewise cardiac images, which, as mentioned above, may be either two-dimensional image slices or three-dimensional volumetric images (though the two-dimensional case will be used for purposes of explanation and illustration herein). FIG. 3 illustrates three representative two-dimensional cardiac images slices 300a, 300b, 300c, each of which includes a region of interest (e.g., a heart chamber) 302a, 302b, 302c for which it is desired to generate a mechanical signal.

In block 204, system 8 segments the blood pool/tissue border for the region of interest in each of the plurality of images. Various segmentation techniques will be familiar to those of ordinary skill in the art including, without limitation, deformable models (e.g., level-set algorithms) and deep network architectures, such as the U-net architecture. See O. Ronnenberger et al, U-net: Convolutional Networks for Biomedical Image Segmentation (MICCAI 2015), which is hereby incorporated by reference as though fully set forth herein. FIG. 3 illustrates the segmented blood pool/tissue borders in images 300a, 300b, 300c as outlines 304a, 304b, 304c, respectively.

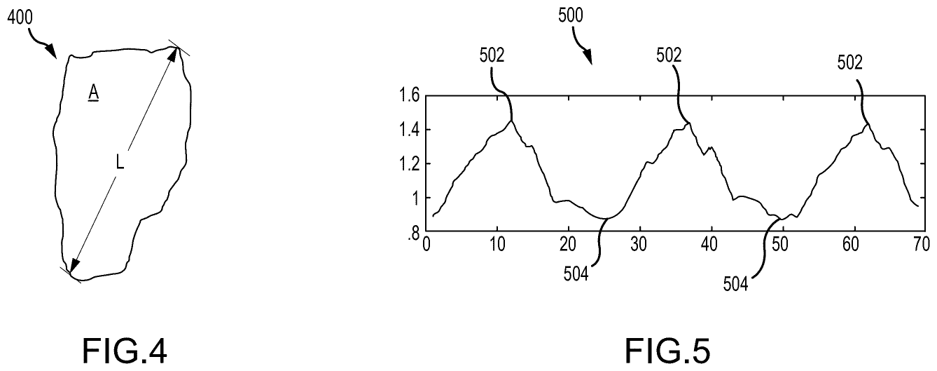

Once segmentation is complete, system 8 detects a blob (that is, a region) corresponding to the area (in two dimensions) or volume (in three dimensions) of the region of interest in block 206. Those of ordinary skill in the art will be familiar with blob detection in the context of computer vision and image processing. By way of illustration, FIG. 4 illustrates a blob 400 corresponding to the segmentation of image 300c.

In block 208, system 8 identifies a cardiac area metric (in two dimensions) or cardiac volume metric (in three dimensions) for each blob detected in block 206. Various metrics are contemplated including, without limitation: blob area (in two dimensions) (shown as “A” in FIG. 4); blob volume (in three dimensions), maximum axial dimension (in two or three dimensions) (e.g., the distance between two points within the blob having the greatest separateion from each other, such as “L” in FIG. 4), Zernike Moments, and other descriptors/quantifications of the shape of an object.

In block 210, system 8 outputs a plot (or trace) of the cardiac metrics identified in block 208 as a function of time. In other words, because the cardiac images are received in block 202 as a time series, their corresponding cardiac metrics (block 208) can be output in the same time series (block 210). A representative trace 500 is shown in FIG. 5 and represents the image-derived cardiac mechanical signal for the region (e.g., a single heart chamber) of interest.

In block 212, system 8 can detect local maxima (502) and local minima (504) in the mechanical signal (500). As those of skill in the art will appreciate, local maxima 502 correspond to diastole, while local minima correspond to systole.

As mentioned above, mechanical signal 500 can be used, either in addition to or in lieu of, an electrical signal, such as an ECG signal, as a gating signal (e.g., for acquisition of data points when using system 8 to create an anatomical model of heart 10 and/or a map of its electrical activity). Unlike an ECG or EGM signal, mechanical signal 500 is naturally synchronized to the heart's movement, which increases the accuracy of temporal characterization of the heart's movement by mitigating delays between the electrical initiation signal and the mechanical initiation of movement.

Further, system 8 can output (e.g., on display 23) a graphical representation of mechanical signal 500. Using a graphical user interface, a practitioner can select points on mechanical signal 500 (e.g., using plots 306a, 306b, 306c) to cause system 8 to display (e.g., on display 23) the corresponding images. For example, if the practitioner wishes to see the image of the region of interest at diastole, the practitioner can select a local maximum (e.g., 308). Similarly, if the practitioner wishes to see the image of the region of interest at systole, the practitioner can select a local minimum (e.g., 310).

As yet another example, mechanical signal 500 can be used to identify all two-dimensional images that were taken at a common cardiac phase. These images can then be assembled, using known techniques, into a three-dimensional volumetric image at that same cardiac phase.

For completeness, the methods described herein may be methods that are embedded within a set of instructions that are comprised within a computer-readable medium or record carrier, or that are comprised within a computer program product. The instructions are such that, when executed by a computer or processor, such as the processor within the electroanatomical mapping system as described herein, or the processor within a mechanical signal generation module as described herein, or a processor of a general-purpose computer system, the computer or processor causes the system or module to perform the methods described herein.

Although several embodiments have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

### Numbered Clauses

1. A method of generating a cardiac mechanical signal, comprising:
receiving a plurality of cardiac images in an electroanatomical mapping system;
for each cardiac image of the plurality of cardiac images, the electroanatomical mapping system identifying a cardiac area metric, thereby identifying a plurality of cardiac area metrics; and
outputting, via the electroanatomical mapping system, a trace of the plurality of cardiac area metrics as a function of time, thereby generating the cardiac mechanical signal.
   2. The method according to clause 1, wherein the electroanatomical mapping system identifying a cardiac area metric comprises, for each cardiac image of the plurality of cardiac images, the electroanatomical mapping system:
segmenting a blood pool/tissue border in the respective cardiac image;
detecting a blob in the segmented respective cardiac image; and
defining the cardiac area metric for the respective cardiac image as a function of a characteristic of the detected blob.
   3. The method according to clause 2, wherein the characteristic of the detected blob comprises an area of the detected blob.
   4. The method according to clause 2 or clause 3, wherein the characteristic of the detected blob comprises a volume of the detected blob.
   5. The method according to clause 2, clause 3 or clause 4, wherein the characteristic of the detected blob comprises a maximum axial dimension of the detected blob.
   6. The method according to any preceding clause, wherein the plurality of cardiac images comprises a plurality of ultrasound images.
   7. The method according to clause 6, wherein the plurality of ultrasound images comprises a plurality of intracardiac echocardiography (ICE) images.
   8. The method according to clause 6 or clause 7, wherein the plurality of ultrasound images comprises a plurality of two-dimensional ultrasound images.
   9. The method according to clause 6, clause 7 or clause 8, wherein the plurality of ultrasound images comprises a plurality of three-dimensional ultrasound images.
   10. The method according to any preceding clause, further comprising:
the electroanatomical mapping system identifying a local minimum in the cardiac mechanical signal as a systole; and
the electroanatomical mapping system identifying a local maximum in the cardiac mechanical signal as a diastole.
   11. The method according to any preceding clause, wherein the plurality of cardiac images comprises a plurality of cardiac images of a single heart chamber.
   12. An electroanatomical mapping system, comprising:
a mechanical signal generation module configured to:
   receive as input a plurality of cardiac images;
   for each cardiac image of the plurality of cardiac images, identify a cardiac area metric, thereby identifying a plurality of cardiac area metrics; and
   output a trace of the plurality of cardiac area metrics as a function of time, thereby generating a cardiac mechanical signal.
      13. The electroanatomical mapping system according to clause 12, wherein the mechanical signal generation module is configured to identify the cardiac area metric for each image of the plurality of cardiac images by:
segmenting a blood pool/tissue border in the respective cardiac image;
detecting a blob in the segmented respective cardiac image; and
defining the cardiac area metric for the respective cardiac image as a function of a characteristic of the detected blob.
   14. The electroanatomical mapping system according to clause 13, wherein the characteristic of the detected blob comprises an area of the detected blob.
   15. The electroanatomical mapping system according to clause 13 or clause 14, wherein the characteristic of the detected blob comprises a volume of the detected blob.
   16. The electroanatomical mapping system according to clause 13, clause 14or clause 15,wherein the characteristic of the detected blob comprises a maximum axial dimension of the detected blob.
   17. The electroanatomical mapping system according to one of clauses 12 to 16, wherein the mechanical signal generation module is further configured to:
identify a local minimum in the cardiac mechanical signal as a systole; and
identify a local maximum in the cardiac mechanical signal as a diastole.
   18. A method of generating a signal representative of variations in a size of an anatomical feature from a plurality of images of the anatomical feature, the method comprising:
receiving, in a mechanical signal generation module, the plurality of images of the anatomical feature;
for each image of the plurality of images, the mechanical signal generation module:
   segmenting a boundary of the anatomical feature in the respective image;
   detecting a blob in the segmented respective image; and
   defining a size metric for the respective image as a function of a characteristic of the detected blob, thereby defining a plurality of size metrics; and
outputting, via the mechanical signal generation module, a trace of the plurality of size metrics as a function of time, thereby generating the signal representative of variations in the size of the anatomical feature.
   19. The method according to clause 19, wherein the size metric is selected from the group consisting of an area of the detected blob, a volume of the detected blob, and a maximum axial dimension of the detected blob.
   20. The method according to clause 18 or clause 19, further comprising at least one of:
identifying a local minimum in the signal as a minimum size of the anatomical feature; and
identifying a local maximum in the signal as a maximum size of the anatomical feature.

What is claimed is:

1. A method of generating a cardiac mechanical signal, comprising:
receiving a plurality of cardiac images in an electroanatomical mapping system;
for each cardiac image of the plurality of cardiac images, the electroanatomical mapping system identifying a cardiac area metric, thereby identifying a plurality of cardiac area metrics; and
outputting, via the electroanatomical mapping system, a trace of the plurality of cardiac area metrics as a function of time, thereby generating the cardiac mechanical signal.

2. The method according to claim 1, wherein the electroanatomical mapping system identifying a cardiac area metric comprises, for each cardiac image of the plurality of cardiac images, the electroanatomical mapping system:
segmenting a blood pool/tissue border in the respective cardiac image;
detecting a blob in the segmented respective cardiac image; and
defining the cardiac area metric for the respective cardiac image as a function of a characteristic of the detected blob.

3. The method according to claim 2, wherein the characteristic of the detected blob comprises an area of the detected blob.

4. The method according to claim 2, wherein the characteristic of the detected blob comprises a volume of the detected blob.

5. The method according to claim 2, wherein the characteristic of the detected blob comprises a maximum axial dimension of the detected blob.

6. The method according to claim 1, wherein the plurality of cardiac images comprises a plurality of ultrasound images.

7. The method according to claim 6, wherein the plurality of ultrasound images comprises a plurality of intracardiac echocardiography (ICE) images.

8. The method according to claim 6, wherein the plurality of ultrasound images comprises a plurality of two-dimensional ultrasound images.

9. The method according to claim 6, wherein the plurality of ultrasound images comprises a plurality of three-dimensional ultrasound images.

10. The method according to claim 1, further comprising:
the electroanatomical mapping system identifying a local minimum in the cardiac mechanical signal as a systole; and
the electroanatomical mapping system identifying a local maximum in the cardiac mechanical signal as a diastole.

11. The method according to claim 1, wherein the plurality of cardiac images comprises a plurality of cardiac images of a single heart chamber.

12. An electroanatomical mapping system, comprising:
a mechanical signal generation module configured to:
receive as input a plurality of cardiac images;
for each cardiac image of the plurality of cardiac images, identify a cardiac area metric, thereby identifying a plurality of cardiac area metrics; and
output a trace of the plurality of cardiac area metrics as a function of time, thereby generating a cardiac mechanical signal.

13. The electroanatomical mapping system according to claim 12, wherein the mechanical signal generation module is configured to identify the cardiac area metric for each image of the plurality of cardiac images by:
segmenting a blood pool/tissue border in the respective cardiac image;
detecting a blob in the segmented respective cardiac image; and
defining the cardiac area metric for the respective cardiac image as a function of a characteristic of the detected blob.

14. The electroanatomical mapping system according to claim 13, wherein the characteristic of the detected blob comprises an area of the detected blob.

15. The electroanatomical mapping system according to claim 13, wherein the characteristic of the detected blob comprises a volume of the detected blob.

16. The electroanatomical mapping system according to claim 13, wherein the characteristic of the detected blob comprises a maximum axial dimension of the detected blob.

17. The electroanatomical mapping system according to claim 12, wherein the mechanical signal generation module is further configured to:
identify a local minimum in the cardiac mechanical signal as a systole; and
identify a local maximum in the cardiac mechanical signal as a diastole.

18. A method of generating a signal representative of variations in a size of an anatomical feature from a plurality of images of the anatomical feature, the method comprising:
receiving, in a mechanical signal generation module, the plurality of images of the anatomical feature;
for each image of the plurality of images, the mechanical signal generation module:
segmenting a boundary of the anatomical feature in the respective image;
detecting a blob in the segmented respective image; and
defining a size metric for the respective image as a function of a characteristic of the detected blob, thereby defining a plurality of size metrics; and
outputting, via the mechanical signal generation module, a trace of the plurality of size metrics as a function of time, thereby generating the signal representative of variations in the size of the anatomical feature.

19. The method according to claim 18, wherein the size metric is selected from the group consisting of an area of the detected blob, a volume of the detected blob, and a maximum axial dimension of the detected blob.

20. The method according to claim 18, further comprising at least one of:
identifying a local minimum in the signal as a minimum size of the anatomical feature; and
identifying a local maximum in the signal as a maximum size of the anatomical feature.
